(19) United States
(12) Patent Application Publication
(10) Pub. No.: US 2018/0300453 A1
(43) Pub. Date: Oct. 18, 2018

(54) Estimation of Descriptive Parameters from a Sample

(71) Applicant: BETH ISRAEL DEACONESS MEDICAL CENTER, INC., Boston, MA (US)

(72) Inventors: Ramy ARNAOUT, Boston, MA (US); Joseph KAPLINSKY, Boston, MA (US)

(73) Assignee: Beth Israel Deaconess Medical Center, Inc., Boston, MA (US)

(21) Appl. No.: 15/750,731

(22) PCT Filed: Aug. 5, 2016
(86) PCT No.: PCT/US2016/045760

§ 371 Date: Feb. 6, 2018

Related U.S. Application Data
(60) Provisional application No. 62/307,352, filed on Mar. 11, 2016.
(60) Provisional application No. 62/202,674, filed on Aug. 7, 2015.

(51) Int. Cl. G06F 19/24; G06F 19/12; C12Q 1/6872; C12N 15/10; G06F 19/22

(52) U.S. Cl. CPC G06F 19/24; G06F 19/12; G06F 19/22; C12N 15/10; C12Q 1/6872

(57) ABSTRACT

An apparatus and method for generating a modified maximum-likelihood description of a system having a parent population of entities of different classes that includes collecting a sample population of the entities and measuring a statistical distribution of the sample population. The method further includes determining a modified maximum-likelihood estimate of the parent population based on the statistical distribution of the sample population and calculating a diversity measure of the estimated parent population to describe the system.

### CROSS REFERENCE TO RELATED APPLICATIONS

This application incorporates by reference in their entireties U.S. Provisional Appl. Nos. 62/202,674, filed Aug. 7, 2015, and 62/307,352, filed Mar. 11, 2016.

### BACKGROUND

### Field

Embodiments herein relate to systems and methods for generating statistical descriptions of a system from a sample.

### Background

Study and analysis of large and complex systems often require sampling. This is true in telecommunications (e.g., signal processing), networking (e.g., the Internet and World Wide Web), physical infrastructure (e.g., roads and power grids), economics (e.g., demographic data), politics (e.g., opinion polling), physics (e.g., astronomy), ecology (e.g., conservation), biology (e.g., genomics, metagenomics, and immunomics), and medicine (e.g., cancer research). However, sampling such systems suffers from inherent bias: the sample is not necessarily an accurate representation of the parent population (i.e., the overall system). For example, the frequency distribution of classes in a parent population generally differs from frequency distribution of classes in a sample of such a system. And, as many descriptive statistics, such as diversity measures (e.g., species richness, Gini-Simpson Index, entropy), depend on the frequency distribution, the statistics of samples often fail to reflect statistics of parent populations of such large and complex systems such as the ones mentioned above.

Sampling may lead to problems in estimating diversity; that is, the diversity of a sample may differ markedly from the diversity of the parent population. With large populations and limited samples, some classes in the parent population, especially rare classes, may not make it into the sample and may thereby go undetected by measurements on the sample. Consequently, sample diversity may underestimate the overall diversity of the parent population. This phenomenon is known in the literature as the “missing-species problem.”

Weighted measures of diversity, such as the entropy or the Gini-Simpson index (see, e.g., Sherwood, A. M. et al. Tumor-infiltrating lymphocytes in colorectal tumors display a diversity of T cell receptor sequences that differ from the T cells in adjacent mucosal tissue. Cancer Immunology, Immunotherapy 62, 1453-1461 (2013); Robert, L. et al. CTLA4 blockade broadens the peripheral T-cell receptor repertoire. Clin Cancer Res 20, 2424-2432 (2014); Arnaout, R. et al. High-resolution description of antibody heavy-chain repertoires in humans. PLoS One 6, e22365 (2011); Laydon, D. J. et al. Quantification of HTLV-1 clonality and TCR diversity. PLoS Comput Biol 10, e1003646 (2014)), on a sample may be less sensitive to missing species than the familiar unweighted measure, species richness, since weighted measures selectively down-weight the contribution of rare classes that are most likely to be missing. This lesser sensitivity suggests that using weighted measures (as a substitute for species richness as descriptive statistics that can be measured on samples) can potentially be a reasonable reflection of the parent population. However, use of weighted measures as a substitute for species richness has two drawbacks. First, it is unclear how much valuable information may be lost by selectively ignoring the (down-weighted) rare classes. Second, for the sample diversity measured by a weighted measure to accurately reflect overall diversity, the frequencies of classes in the sample must reflect the frequencies of the classes in the parent population. But this may be complicated by sampling noise for many class frequencies. Consequently, depending on the specific measure and frequency distribution, missing species may still cause appreciable error in an overall diversity estimate, using weighted measures.

Past attempts at estimating the number of missing species have had limitations. Fisher's gamma-Poisson mixture method, a parametric method, involves a divergent sum that can result in large uncertainties in the number of missing species and thereby in the measurement of the diversity of the parent population using species richness as the diversity measure. Moreover, because Fisher's method does not output overall class sizes, it does not produce weighted measures. A nonparametric method by Chao (the Chao estimator (CE)) (see, e.g., Chao, A. & Lee, S. M. Estimating the number of classes via sample coverage. Journal of the American Statistical Association 87, 210-217 (1992); Chao, A. Nonparametric-estimation of the number of classes in a population. Scandinavian Journal of Statistics 11, 265-270 (1984)), based on the Good-Turing estimator, avoids divergent sums and has been widely used in ecology; however, like Fisher's method, it provides only species richness. So does the method of extrapolating from curve fitting, which in addition is somewhat arbitrary. Nonparametric approaches such as those by Norris and Pollock (NP) (see, e.g., Norris, J. L. & Pollock, K. H. Nonparametric MLE under two closed capture recapture models with heterogeneity. Biometrics 52, 639-649 (1996); Norris, J. L. &. Pollock, K. H. Non-parametric MLE for Poisson species abundance models allowing for heterogeneity between species. Environmental and Ecological Statistics 5, 391-402 (1998)) and Wang and Lindsay (WL) (see, e.g., Wang, J. P. Z. & Lindsay, B. G. A penalized nonparametric maximum likelihood approach to species richness estimation. Journal of the American Statistical Association 100, 942-959 (2005)) use maximum likelihood to provide additional measures, but existing implementations either do not scale to complex parent populations which have many classes, risk overfitting or getting trapped in local maxima, or make restrictive assumptions about the size distribution of the parent population and therefore are not generalizable. Moreover, because a higher-likelihood fit can often be had by adding more small classes, existing maximum-likelihood approaches yield estimates that may overestimate diversity by orders of magnitude or be entirely unbounded—that is, they may find that the best estimate of diversity in the overall population is infinity.

### BRIEF SUMMARY

Accordingly, there is a need for a method and apparatus to reconstruct a parent population, with error bars, from a sample without making assumptions about the frequency distribution of classes in the parent population in order to improve accuracy in the description and analysis of the parent population. More specifically, there is a need for non-obvious and non-trivial innovations relative to traditional maximum-likelihood methods to satisfy this need.

According to an embodiment, a method for generating a modified maximum-likelihood description of an individual's adaptive immune system (i.e., B and T cells; henceforth simply “immune system”) having a parent population of immune cells of different clones of immune cells (where “clone” denotes cells with a common B- [or T-]cell progenitor) includes collecting a sample population of these immune cells from the individual and measuring a statistical distribution of the sample population of immune cells using a nucleic-acid sequencing device. The method further includes determining a modified maximum-likelihood estimate of the parent population based on the statistical distribution of the sample population to produce an estimated parent population and calculating a diversity measure of the estimated parent population, where the diversity measure is a representation of the modified maximum-likelihood description of the individual's immune system. The determining of the modified maximum-likelihood estimate includes initializing the fit of a statistical model of the parent population based on the sample population, and iteratively optimizing the statistical model until best-fit parameters of the statistical model can no longer be improved without crossing a sampling-noise threshold.

According to another embodiment, an apparatus configured to generate a modified maximum-likelihood description of an individual's immune system having a parent population of immune cells of different clones of immune cells includes a measurement unit and a processing unit. The measurement unit may be configured to receive a sample population of immune cells from the individual and compute a statistical distribution of the sample population of immune cells. The processing unit may be configured to determine a modified maximum-likelihood estimate of the parent population based on the statistical distribution of the sample population to produce an estimated parent population. To determine the modified maximum-likelihood estimate of the parent population, the processing unit may be further configured to initialize a statistical model of the parent population based on the sample population and iteratively optimize the statistical model until best-fit parameters of the statistical model can no longer be improved without crossing a sampling noise threshold.

In another embodiment, a computer program product stored on computer-readable media includes a set of instructions that, when executed by a computing device, perform steps including measuring a statistical distribution of a sample population of immune cells from an individual's immune system having a parent population of immune cells of different clones of immune cells. The steps further include determining a modified maximum-likelihood estimate of the parent population based on the statistical distribution of the sample population to produce an estimated parent population and calculating a diversity measure of the estimated parent population, where the diversity measure is a representation of a modified maximum-likelihood description of the individual's immune system.

Further features and advantages, as well as the structure and operation of various embodiments, are described in detail below with reference to the accompanying drawings. It is noted that the specific embodiments described herein are not intended to be limiting. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate the present invention and, together with the description, further serve to explain the principles of the present invention and to enable a person skilled in the relevant art(s) to make and use the present invention.

FIG. 1 illustrates a flowchart for generating a statistical description of a population, according to an embodiment.

FIG. 2 illustrates a flowchart for generating a modified maximum-likelihood estimate of a parent population, according to an embodiment.

FIG. 3A illustrates example comparison plots for different diversity measures and FIG. 3B illustrates speed and accuracy relative to older methods for the diversity measure known as species richness.

FIGS. 4A-4G illustrate example comparison plots for different diversity measures.

FIG. 5 illustrates example comparison plots between best-fit and observed sample distributions for in silico (e.g., generated on a computer) distributions.

FIG. 6 illustrates example speed and accuracy plots relative to older methods for additional diversity measures (labeled ¹D, ²D, and ^{∞}D).

FIG. 7 illustrates example comparison of accuracy for different number of starting points used in the example method of FIGS. 1 and 2.

FIGS. 8A-8D illustrate example methods for determining error profiles.

FIG. 9 illustrates example best-fit and observed distributions for different ex vivo (i.e., real-world) example populations (immune repertoires).

FIG. 10 illustrates a block diagram of an apparatus for generating a statistical description of a system, according to an embodiment.

FIG. 11 illustrates a block diagram of a computer system in which embodiments of the present invention, or portions thereof, may be implemented.

The present disclosure will now be described with reference to the accompanying drawings. In the drawings, like reference numbers generally indicate identical or similar elements. Additionally, generally, the left-most digit(s) of a reference number identifies the drawing in which the reference number first appears.

### DETAILED DESCRIPTION OF THE INVENTION

This specification discloses one or more embodiments that incorporate the features of this invention. The disclosed embodiment(s) merely exemplify the invention. The scope of the invention is not limited to the disclosed embodiment(s). The invention is defined by the claims appended hereto.

The embodiment(s) described, and references in the specification to “one embodiment,” “an embodiment,” “an example embodiment,” etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

Embodiments of the present invention may be implemented in hardware, firmware, software, or any combination thereof. Embodiments of the present invention may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices, and others. Further, firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc.

It is to be understood that the phraseology or terminology herein is for the purpose of description and not of limitation, such that the terminology or phraseology of the present specification is to be interpreted by those skilled in relevant art(s) in light of the teachings herein.

The following terms have the meanings given below, unless specified otherwise, to describe various embodiments of the present disclosure. As used herein:

The term “entities” describe the elemental components of a system of interest.

The term “class” refers to a group of entities of a given type.

The term “parent population” describes a sum total of all entities in all classes in the system.

The term “sample” or “sample population” describes a subset of the parent population that may be produced by random sampling.

The terms “observed entities” and “observed classes” refers to respective entities and classes in the sample.

The term “missing classes” or “missing species” refers to classes that are present in the parent population but not observed in the sample.

The term “size of the class” or “class size” describes the number of entities in a class. In embodiments, the size of a class may differ between the parent population and the sample, since the number of entities differs between the parent population and the sample. It should be noted that a sample cannot contain more entities than the parent population from which it derives.

The term “clone size” refers to the number of cells that make up a clone. A clone made up of a single cell has clone size 1, while a clone made up of a million cells has clone size one million. A clone is an example of a class.

The term “class size distribution,” “size-frequency distribution of classes” or “frequency distribution of classes” in a population describes the histogram of the number of classes of each size. In embodiments, the frequency distribution differs between the parent population and the sample due to inherent features of sampling processes.

The term “parent distribution” or “overall distribution” refers to a class size distribution in a parent population.

The term “clone size distribution” refers to the number of clones of each size.

### Overview

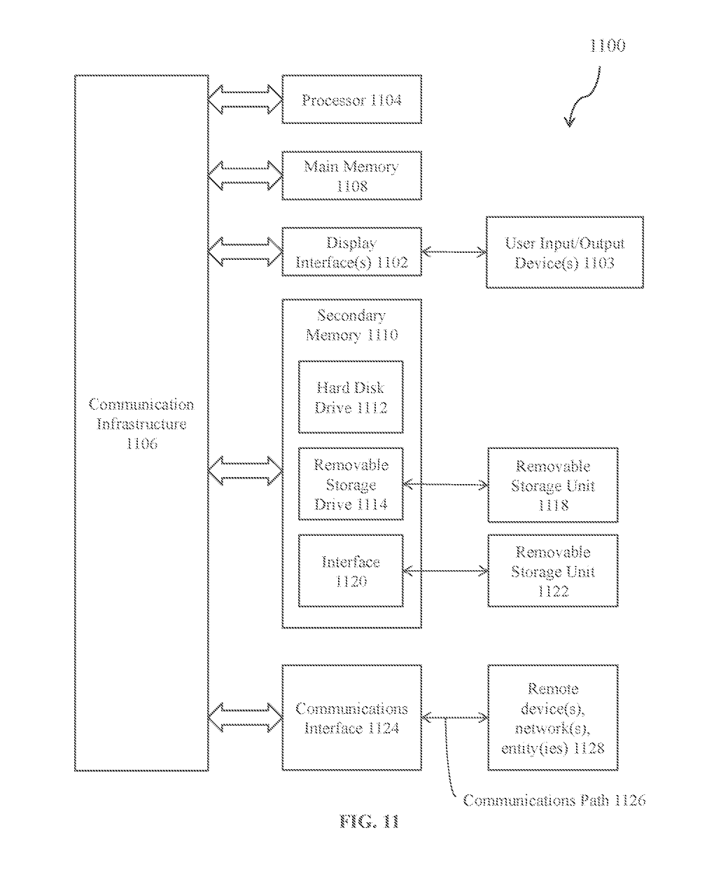

This disclosure provides example methods (e.g., method 100) and apparatus (e.g., FIGS. 10-11) for reconstruction of a parent population, with error bars, from a sample without making assumptions about class sizes or the frequency distribution of classes in the parent population, for estimation of any diversity measures, and for statiscally reliable comparison between samples, including between systems and in the same system over time, for complex populations/systems. The example methods determine a maximum likelihood estimate (MLE) for parameters of a model describing the sampling distribution from the parent population. The form of the model may have an immediate interpretation in terms of the class size distribution of the parent population.

These example methods and apparatus may provide for one or more parent populations of a system (i) the overall class size distribution; (ii) the diversity of the overall population as measured by species richness, entropy or any other diversity measure such as or related to those in the framework described by Hill (see, e.g., Hill, M. O. diversity and evenness—unifying notation and its consequences. Ecology 54, 427-432 (1973)), with error bars; (iii) the number of missing species, with error bars; (iv) the minimum detected class size; (v) the diversity of the sample population, for comparison to overall diversity and (vi) a resampling of the overall distribution for comparison to the sample and plots thereof.

These example methods provide several advantages over current methods of describing and analyzing large and complex systems. For example, to avoid dependence on initial conditions or becoming trapped in local maxima, these example methods may include scanning a number of initial conditions in each iteration of an algorithm. Such scanning may produce substantially better estimates of overall class sizes, missing species and diversity measurements of a parent population compared to that produced by known methods. These example methods may further include optimizing the average of the two best fits in each iteration of the algorithm and checking to prevent overfitting due to sampling noise. These example methods are widely applicable as no assumptions are made about the overall class size distribution. Also, these example methods provide improvement over previous maximum-likelihood models in processing speed and in avoiding unbounded uncertainties, for example, regarding bounds on overall diversity estimates. The processing speed of these example methods (e.g., using commercially available computer processing hardware, using conventional personal computers for populations of a billion entities and tens of millions of classes) may be in a range from one second to one minute, which is faster than processing speeds of previous methods (e.g., WL, NP). Yet another advantage of these example methods over known methods is in their use of both a noise threshold and the (corrected) Akaike information criterion, which may provide tighter bounds, rejecting additional classes unless their expected contribution to the sample rises above a sampling-noise threshold value, and which outweighs the penalty for adding more parameters and avoids overfitting.

### Method for Generating a Statistical Description of a System According to an Embodiment

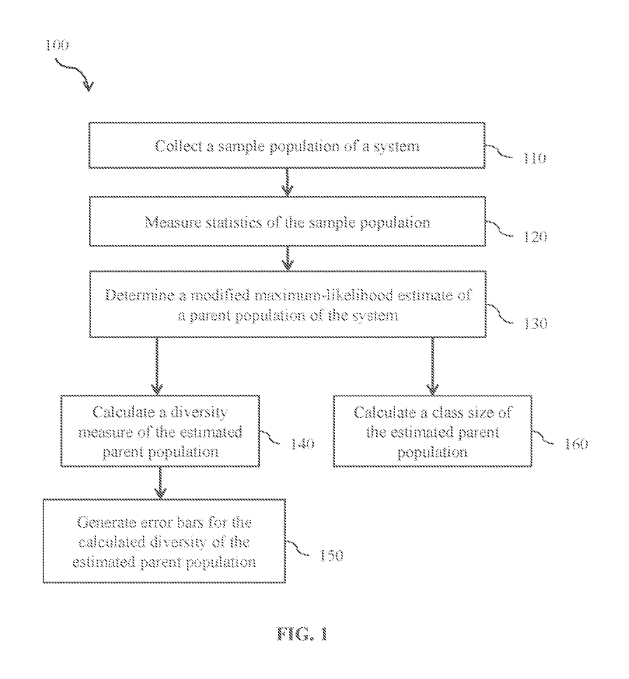

FIG. 1 illustrates a method 100 for generating a statistical description of a system, according to an embodiment. Method 100 may be referred to as Recon in examples of this disclosure. For purposes of explanation, the invention is described in the example environment of the B or T cells of the adaptive immune system of an individual (e.g., a human or a mouse). In this environment, method 100 may be used to generate one or more descriptive statistics of the immune system. The generated one or more descriptive statistics may be one or more diversity measures of the parent population that is the overall set of these cells in an individual, according to an example. Such diversity measures of an individual's immune cell parent population may be both clinically important and intrinsically interesting as a measure of complexity in the adaptive immune system. For example, determining the diversity of a B- or T-cell population of the adaptive immune system of an individual is both clinically important and a key measure of immunological complexity. Method 100 may be used to find the B- or T-cell clone-size distribution in the individual that is most likely to give rise to the clone-size distribution that is observed in the sample. Method 100 may then be used to calculate the overall diversity according to any diversity measure in the Hill framework or related diversity measure, from the sample distribution.

Method 100 may be based on an expectation-maximization (EM) algorithm and may include iterative refining of a rough approximation of an overall distribution until no further improvement can be made without overfitting to determine a modified maximum-likelihood estimate (MLE) of a parent population. In a first iteration to determine the modified MLE, method 100 may include assuming a parent distribution in which classes are all the same size, taken from a mean of the class size distribution of a sample distribution from the parent population. Method 100 further includes estimating the number of missing species by calculating the expected class size distribution for a (Poisson) sample of the parent distribution and applying the Horvitz-Thomson estimator (see, e.g., Armitage, P. & Colton, T. Encyclopedia of biostatistics, Edn. 2^{nd}. (John Wiley, Chichester, West Sussex, England ; Hoboken, N.J.; 2005)). The class size of the parent distribution may then be fitted using maximum likelihood and the number of missing species recalculated. These steps may then be repeated until a self-consistent number of missing species is obtained, which may complete the first iteration to determine the modified MLE, yielding a uniform parent distribution that is most likely to give rise to the sample distribution from the parent population.

In a second iteration to determine the modified MLE, method 100 may include refining this uniform parent distribution by adding a second class size. The refinement process may include estimating the number of missing species for this new two-size distribution, fitting the two class sizes and their relative frequencies by maximum likelihood, and repeating these steps until there is no further improvement, yielding a two-class-size parent distribution that is most likely to give rise to the sample distribution. In subsequent iterations to determine the modified MLE, method 100 may include further refining of the parent distribution by adding class sizes, refitting as in the second iteration, and iterating until no more class sizes can be added without overfitting, and using a corrected Akaike information criterion as a stop condition.

As shown in FIG. 1, method 100 may start at step 110 where a sample population of entities is collected from a parent population of entities, according to an embodiment. For example, a sample population of immune cells may be collected from an individual's immune system. This collection process may involve drawing and processing of a blood sample from the individual to extract the sample population of immune cells.

At step 120, statistics such as the frequency distribution for the class size distribution of the sample population are measured, according to an embodiment. For example, statistics such as the clone-size distribution of the sample immune cell population may be measured. This measurement may be performed using a nucleic-acid sequencing device or a mass spectrometer, according to an example. An assumption may be made that each class (e.g., clone) in the individual contributes cells to the sampled population according to a Poisson distribution. This may be true if, according to an example, (i) clones are well mixed in the blood or evenly distributed in the tissue being sampled, (ii) the parent population is sufficiently large that the Poisson estimate for the probability of, for example, a singleton contributing >1 cell is negligible and (iii) no single clone is a large fraction (˜30% or more) of the parent population.

At step 130, a modified maximum-likelihood estimate of a parent population is determined, according to an embodiment. For example, a modified MLE of the immune cell parent population in the individual's immune system may be determined. In an example, the immune cell parent population may be represented by its clone-size distribution. This modified maximum-likelihood estimation of the immune cell parent population maximizes the probability of observing the measured sample population distribution of step 120 in a sample of the estimated immune cell parent population, subject to the constraints of a sampling-noise threshold and the corrected Akaike information criterion. In an example of this embodiment, this modified maximum-likelihood estimation may be based on the EM algorithm. This estimation method may involve finding a modified MLE for parameters of a model representing the parent population and/or class size distribution of the parent population of the system. In an example, the parent population may be represented by a probability distribution model, such as, but not limited to, a mixed Poisson model. This model may predict the contribution of each class (e.g., clone) in the parent population to the sample population by equation (1) below,

$${p}_{i} = {\sum }_{j} {w}_{j} Poisson ( i ; {m}_{j} ) = {\sum }_{j} {w}_{j} \frac{{m}^{i} exp ( - {m}_{j} )}{i !} \qquad ( 1 )$$

where wⱼ are weights and mⱼ are Poisson parameters. The weights wⱼ may give the proportion of classes in the parent population with class size j. The parameters mⱼ may give the mean number of entities a class of size j may contribute to a sample of the parent population and may be referred to as means. The parameters wⱼ and mⱼ may provide a description of the class size distribution in the parent population by equation (2) below,

class size j in parent population=number of entities in parent population x mⱼ/sample size (2)

The notation for class size used here is nᵢ, where i indexes the class size, and nᵢ denotes the number of classes of that size, n₁ may denote the number of classes represented in the sample by a single entity, while n₂ may denote the number of classes represented by two entities. The number of missing classes that are present in the parent population but may be missing from the sample population is represented by n₀, as these missing classes are represented by zero entities in the sample.

If there are k different sizes in the parent population, so that the index j ranges from 1 to k, then there are a total of 2k-1 independent parameters, consisting of k independent sizes mj and k-1 independent weights wj, which sum to 1. Assuming that the sample comes from a well mixed parent population, such as blood, this may give rise to a sampling distribution of equation (3):

$$P ( {n}_{0} , {n}_{1} , {n}_{2} … ) = ( \frac{{n}_{total} !}{( {n}_{0} ! n 1 ! {}_{1}^{}{n}{}_{2}^{} ! … )} ) \times ( {p}_{0}^{{n}_{0}} , {p}_{1}^{{n}_{1}} , {p}_{2}^{{n}_{2}} … ) \qquad ( 3 )$$

where nₜₒₜₐₗ is the sum of all n_{i.}

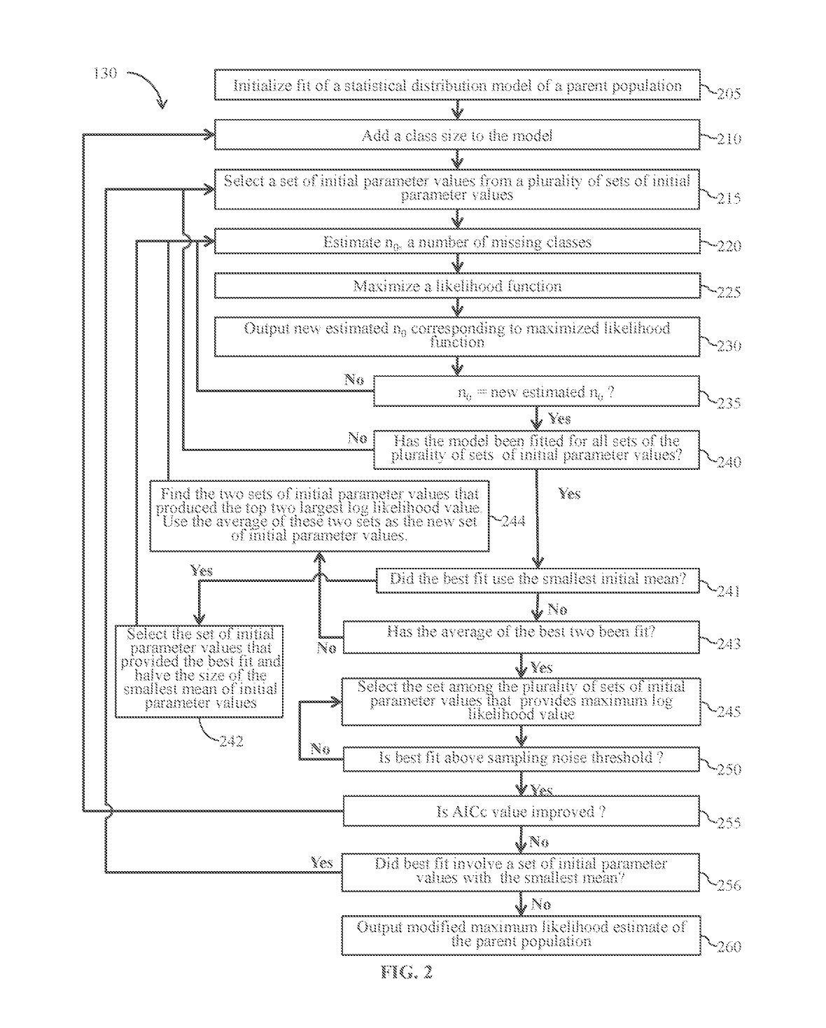

Step 130 may include sub-steps 205-260, as illustrated in FIG. 2, that are involved in determining the modified maximum-likelihood estimate of the parent population, according to an embodiment.

At sub-step 205, a fit of a statistical distribution model of the parent population of the system is initialized, according to an embodiment. In an example, the statistical distribution model may be the mixed Poisson model discussed above. The step of initializing the fit may involve initializing a parent population having class sizes all of the same size. This size may be based on the mean size of classes in the sample population. This step may also involve initializing the number of missing classes using equation (4) below

n₀=n_{obs}/(1−p₀) (4)

where n_{obs} may be the sum of nᵢ for 0>i>tau in the sample population, and tau is some threshold above which sampling noise is assumed to be small (e.g., 30, in an embodiment). Sub-step 205 may further include splitting the class size distribution of the sample population into large and small classes. The mean size of all classes of the sample population contributing to the fit (i.e., clones contributing less than 30 cells, e.g., in some embodiments) may be calculated. This calculation may be used to set the scale for initial guesses of classes sizes in the following sub-step 210. The initial parameters for the fit of the model may be set to empty lists of weights and means, and may be recorded as the current best fit.

At sub-step 210, a new class size is added to the model in such a way that the new distribution maximizes the log likelihood function P given by equation (3) above, according to an embodiment. This step of adding a class size may involve adding a new distinct class size k to the parent population model.

At sub-step 215, a set of initial parameter values from a plurality of initial parameter values is selected, according to an embodiment. In an example of this embodiment, the set of initial parameter values may include values of wⱼ and mⱼ e.g., values in Tables 1 and 2, respectively below), which may be calculated, for example, based on the statistics of the sample population measured at step 120. This step of selecting the set of initial parameter values may involve selecting the values of wⱼ and mⱼ that correspond to the new class size added in sub-step 210.

In an embodiment, except on the first iteration of the fit, the weight of the new class size may be selected from a list of starting weights in Table 1 below. On the first iteration of the fit the newly added class size may be the only class size, so the weight is 1.0. The mean for the new population may be calculated by selecting a starting scale factor in Table 2 below and multiplying by the mean size of the small clones.

TABLE 1
Starting weights

0.05
0.128
0.207
0.286
0.364
0.443
0.571
0.6

TABLE 2
Starting scale factors

0.05
0.225
0.4
0.575
0.75
0.925
1.1

At sub-step 220, a number of missing classes n₀ is estimated, according to an embodiment. This step may involve recalculating the number of missing classes using equation (4) above and the new pᵢ that may be recalculated using equation (1) and corresponding wⱼ and mⱼ values selected in sub-step 215.

At sub-step 225, a likelihood function is maximized, according to an embodiment. In an example, a likelihood function P may be represented by equation (3) given above, where the value of may be based on the number of class sizes in the model after sub-step 210, and the value of pᵢ may be calculated based on the values of wⱼ and mⱼ selected at sub-step 215. This maximizing step may involve maximizing the logarithm of a likelihood function P for the total number of class sizes in sub-step 210, the selected wⱼ and mⱼ values in sub-step 215, and the estimated number of missing classes in sub-step 220. This sub-step 225 may record the log likelihood value for which the parameters of the model, such as wⱼ and mⱼ and number of missing classes are maximized. The log likelihood value may be recorded in a look-up table in, for example, a storage unit that may be accessed later.

At sub-step 230, a new estimated number of missing classes n₀ is recorded, according to an embodiment. This step may involve recalculating the number of missing classes using equation (4). This new estimated number of missing classes may be the number of missing classes that corresponds to the maximized log likelihood function of sub-step 225.

At sub-step 235, the estimated numbers of missing classes of sub-steps 220 and 230 are compared, according to an embodiment. In an embodiment, if the new estimated value of n₀ of sub-step 230 is equal to the old value of n₀ of sub-step 220, then method 100 moves to sub-step 240. In an embodiment, if the new estimated value of n₀ of sub-step 230 differs from the old value of n₀ of sub-step 220, sub-steps 220-235 may be repeated using the new estimate and starting from the initial parameter values given by the fit for the old n₀ estimate until these numbers are substantially equal. This sub-step may ensure that a self-consistent estimate for n₀ is obtained for a set of initial parameter values that maximize likelihood. This set of initial parameter values may be added to a list of possible best fits in a look-up table in, for example, a storage unit that may be accessed later.

At sub-step 240, a check is performed to determine whether the model is fitted for all sets of the plurality of sets of initial parameter values, according to an embodiment. This step may involve accessing the look-up table to determine if a log likelihood value for each set of the plurality of sets of initial parameter values has been stored. Sub-steps 215-240 may be repeated until a maximum likelihood function P for all sets of the plurality of sets of initial parameter values has been determined and added to the list of possible best fits.

At sub-step 241, a check is performed to determine whether the best fit from sub-step 240 (e.g., the fit with the highest log likelihood) started from the smallest initial mean mⱼ, according to an embodiment. If the best fit started from the smallest initial mean mⱼ then the starting weights wⱼ and means mⱼ that lead to the best fit may be taken, and the smallest mean mⱼ may be halved in value at sub-step 242. These starting weights wⱼ and means mⱼ may then be passed on to sub-step 220. Sub-steps 220-242 may be repeated until the best fit is obtained that did not start from the smallest initial mean mⱼ.

At sub-step 243, a check is performed to determine whether a fit has been performed with an average of the starting weights wⱼ that led to the two best fits from sub-step 240 (e.g., the two fits with the top two highest log likelihood), according to an embodiment. If such a fit has not been performed, the starting weights wⱼ and means mⱼ that lead to the best two fits may be taken and averaged together to produce the best average starting weights at sub-step 244. These starting weights wⱼ and means mⱼ may then be passed on to sub-step 220.

At sub-step 245, a set among the plurality of sets of initial parameter values is selected that corresponds to maximum log likelihood value, according to an embodiment. This step may involve accessing the look-up table to determine the largest value among the maximum log likelihood values recorded for each set of the plurality of sets of initial parameter values. The parameters of the model corresponding to this largest value may be selected in this step and may be considered as being the best fit parameters of the model.

At sub-step 250, a check is performed to determine whether the best fit parameters of the previous sub-step are above a sampling noise threshold value, according to an embodiment. The sampling noise threshold value may be set based on the contribution of the smallest class size to classes that are observed once in the sample population. In an embodiment, the sampling noise threshold value may be set at three standard deviations, where the standard deviation is of the contribution from the larger class size to the sampling noise and the standard deviation may be estimated as the square root of the expected contribution from the larger class size to the sampling noise. This sub-step may contribute to the modification in the modified maximum-likelihood method. Sub-steps 245-250 may be repeated until best fit parameters are found above the predefined sampling noise threshold value.

At sub-step 255, a check is performed to determine whether the corrected Akaike information criterion (AICc) (i.e., a measure of the relative quality of a statistical model) is improved, according to an embodiment. Sub-steps 210-255 may be repeated until no improvement (or no substantial improvement) in the AICc condition is observed.

At sub-step 256, a check is performed to determine whether the best fit of the previous sub-step involved a set of initial parameter values with the smallest mean, according to an embodiment. Sub-steps 215-256 may be repeated until the best fit involves a set of initial parameter values with the smallest mean.

At sub-step 260, a modified MLE of the parent population is output if no improvement (or no substantial improvement) is observed in sub-step 255. In that case, the best fit involves a set of initial parameter values with the smallest mean in sub-step 256.

Thus, according to an embodiment, determining the modified MLE of the parent population comprises iteratively optimizing a statistical fit based on agreement with the sample distribution, adding parameters as needed until no further improvement can be made without overfitting (e.g., crossing a sampling noise threshold and using the corrected Akaike information criterion as a stopping condition).

Referring back to FIG. 1, at step 140, diversity measures of the modified maximum-likelihood estimated parent population are determined. In an example of this embodiment, diversity measures such as species richness, Shannon entropy, Gini Simpson index, Berger-Parker index, or other possible measures in or related to Hill's extensive diversity framework may be determined for the estimated parent population.

At step 150, error bars (i.e., graphical or numerical representation of variability in diversity estimates) for the calculated diversity measures are generated, according to an embodiment. In an example of this embodiment, step 150 may be an optional step. This step of generating error bars may involve generating an in silico parent population, i.e., a known parent population, with known diversity, taking samples of this known distribution at systematically increasing sample sizes, determining modified MLE of the known parent population, and determining diversity measures of the estimated known parent population for each sample size using, for example, steps 130, 205-260, and 140 of method 100 discussed above. These steps may result in a reference table for how error may fall with increasing sample size for a given level of diversity.

The error bar generation may further include comparing the diversity measures of the estimated parent population for the sample for which error bars are desired with the diversity measures of the estimated parent population for samples of known diversity. Comparing may include looking up in the reference table the largest and smallest diversity measure values that are consistent with the diversity measure values of the estimated parent population or interpolating between the looked-up values. These upper and lower bounds may define the desired error bar (or error bars). Such error bars may help in drawing reliable conclusions about differences in the diversity measures of the estimated parent population from differences between samples. An error bar may define the range of overall diversity measure values that, given the inevitable error involved in reconstructing parent distributions from samples of a given size, is consistent with the estimated parent population.

At step 160, a class size of the estimated parent population is calculated based on a class size of the sample for a type of entity. In an example of this embodiment, step 160 may be an optional step. This step may include using the modified maximum-likelihood estimated parent population to replace the observed class sizes (i.e., class sizes of the sample population) with the class sizes in the parent population that would be expected based on random sampling to give rise to the observed class sizes. This step may be referred to as the resealing of the class sizes of the sample population, and is calculated as follows:

rescaled_class_size=sumⱼ (muⱼ*wⱼ*poisson(muⱼ, obs_class_size)/sumⱼ(wⱼ*poisson(muⱼ, obs_class_size))

where j may index the class sizes in the modified maximum-likelihood estimated parent population, poisson(mu, n) is a poisson distribution function with mean mu, evaluated at n, and obs_class_size is the size of the class observed in the sample.

The following equation may be used to calculate the relative sizes of two classes in the parent population based on the relative sizes of those two classes in the sample population:

size of class 1 in parent population/size of class 2 in parent population=rescaled class size of class 1/rescaled class size of class 2

It should be noted that resealing may give information about averages; it is possible that a larger class will have fewer, or the same number, of entities in the sample as a smaller class.

It should be noted that method 100 may not include all operations shown or perform the operations in the order shown.

According to an embodiment, method 100 may be implemented using a computer program written in Python based source code as provided, for example, in Appendix A of this specification. Method 100 may be referred as Recon in this disclosure.

### EXAMPLES

Provided herein are various examples of generating descriptions of a system a sample of the system using methods similar to that described above with reference to FIGS. 1 and 2. These examples are not intended to limit the scope or spirit of the invention in any way but are illustrative of the principles of operation of the invention.

### Experiment #1

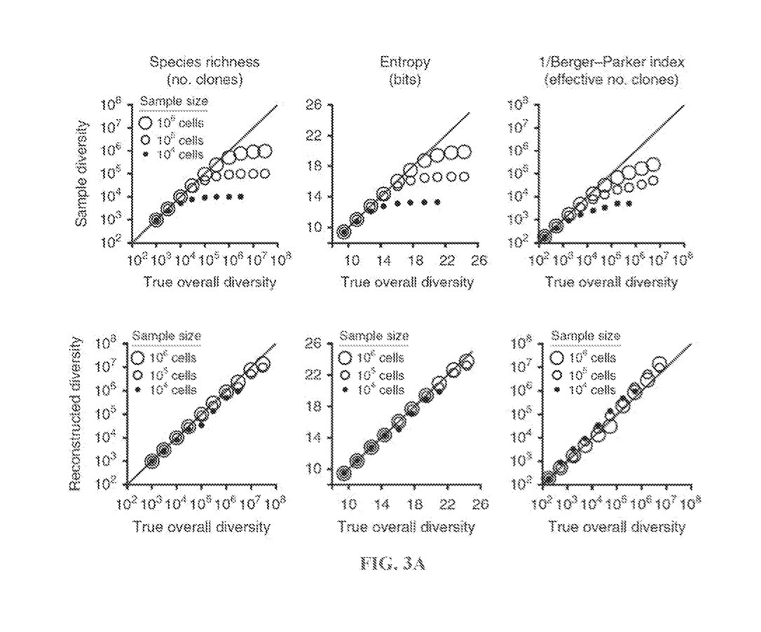

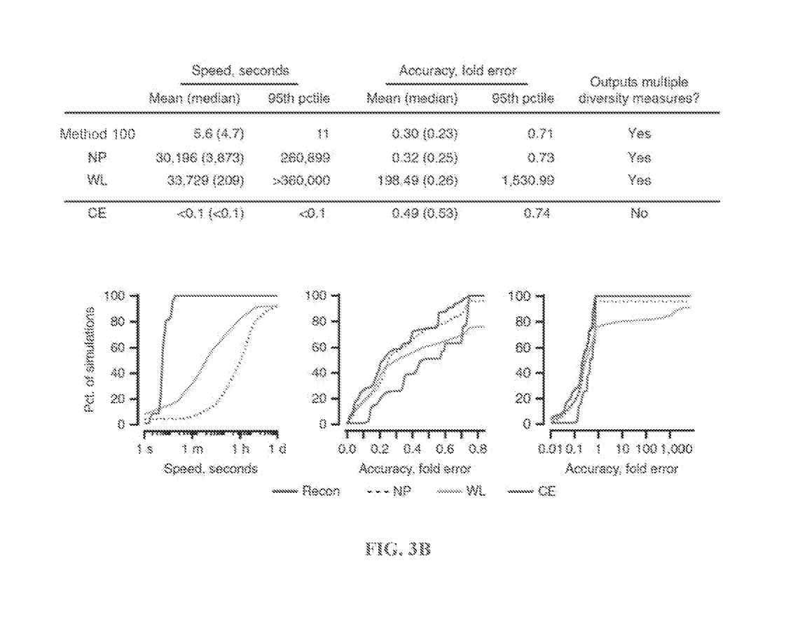

A study was performed with in silico repertoires that spanned nearly five orders of magnitude of overall diversity (300 to 10 million clones) and a wide range of clone-size distributions: from steep, that is, dominated by small clones, to flat exponentials; reciprocal—exponential distributions that derive from a generative model; and multiple bimodal distributions of small and large clones, 1,711 in all, with and without simulated experimental noise. These repertoires served as gold standards. For each in silico repertoire, a known number of cells were sampled, for coverage ranging from 0.01× to 10×. Coverage is the number of cells in the sample divided by the number of clones in the overall repertoire. For each sample, the overall repertoire (i.e., the parent population) was reconstructed and the diversity of the reconstructed overall repertoire determined using, for example, method 100 as described above with reference to FIGS. 1 and 2. The diversity of the sample repertoire (“sample diversity”) was compared to the overall diversity, and the diversity of the reconstructed overall repertoire was also compared with the overall diversity, as illustrated in FIG. 3A. FIG. 3A illustrates comparison between sample diversity and overall diversity (top) and between the estimate of overall diversity using method 100 and overall diversity (bottom) for three different sample sizes—10,000 cells (filled circles), 100,000 cells (small open circles) and 1 million cells (large open circles) for samples taken from a representative gold-standard distribution without noise (i.e., random error on the counts of clone-size frequencies in the sample). The diversity was measured according to species richness, entropy, and the inverse Berger-Parker Index as shown in FIG. 3A.

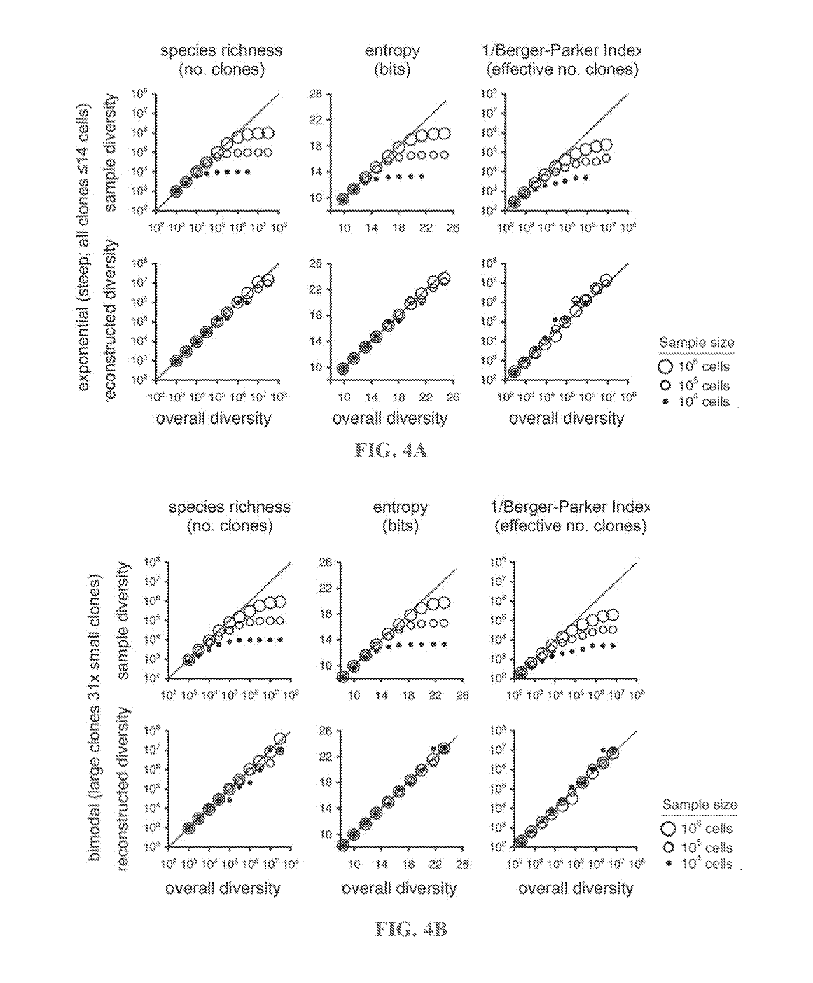

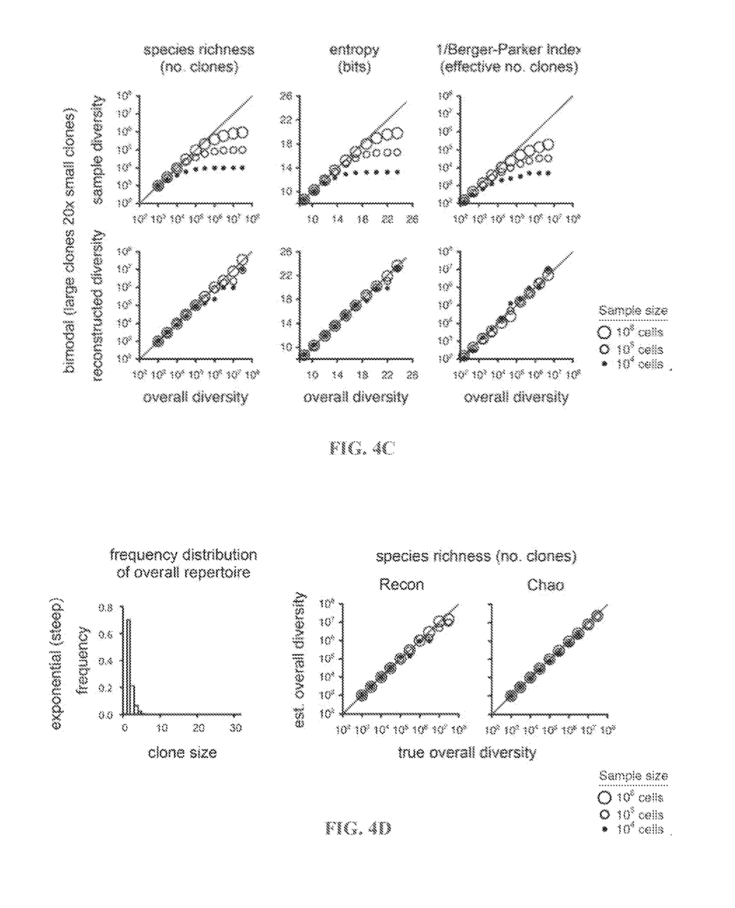

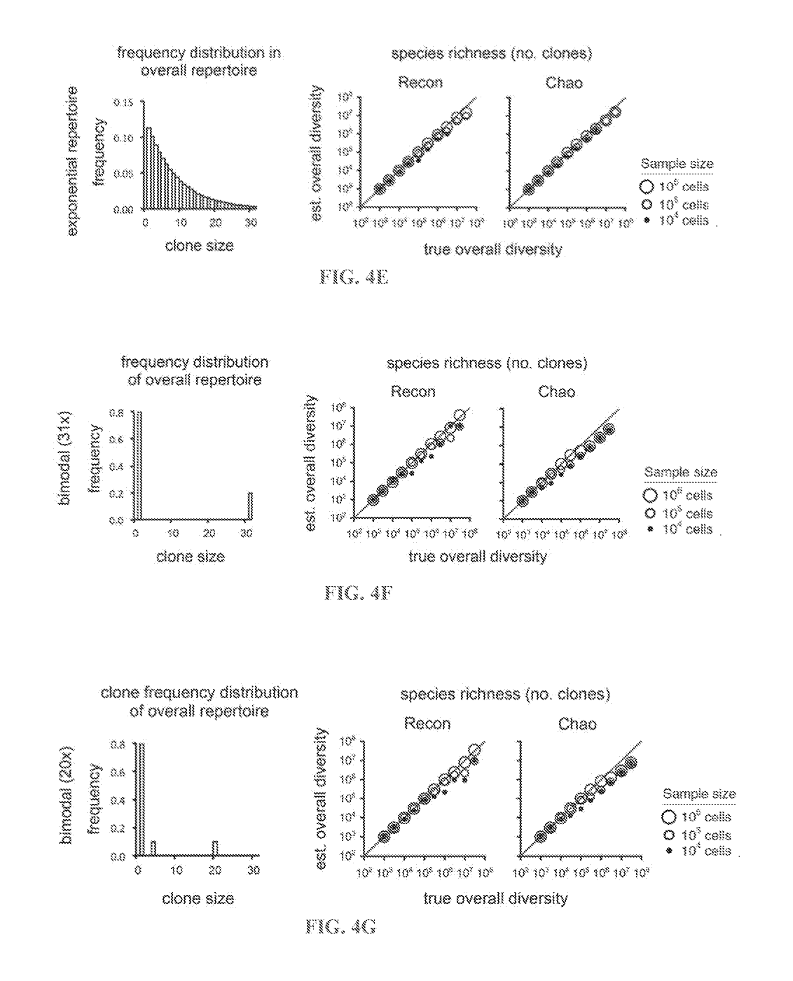

In this study, to illustrate the extent of the problems method 100 may solve, sample diversity was compared with overall diversity as shown in FIG. 3A. For a given sample size, higher overall diversity may mean lower clonal coverage (the number of cells in the sample per clone in the overall repertoire). For each repertoire, the error, defined as the difference between sample and overall diversity, may grow as coverage falls below 1×, because samples cannot have more clones than cells. Consequently, for species richness, sample diversity underestimated true diversity by 50% at 1× coverage, 10 fold at 0.1× coverage, and 30 fold at 0.03× coverage. The weighted measures (entropy and inverse Berger-Parker index) performed little better, even for the flattest clone-size distributions, which may be partly due to the absence of clones large enough to dominate these repertoires (e.g., leukemic clones; FIGS. 3A and 4A-4C). Thus, this study showed sample diversity may be generally an unreliable proxy for true diversity below 1× coverage in the absence of dominant clones.

In contrast, estimates of overall diversity found using method 100 showed excellent agreement with true diversity across the range of diversity measures, even at <1× coverage as shown in FIG. 3A, lower panels, and FIGS. 4A-4G. FIGS. 4A-4G show Recon diversity versus other estimates showing fits to additional gold standard repertoires plotted as in FIG. 3A. FIGS. 4A-4C illustrate comparisons of sample diversity (top) to Recon diversity (bottom) plotted as in FIG. 3A for: a steep exponential clone size distribution, a bimodal distribution in which the overall distribution contains a population of small clones and a population 31 times as large, and a bimodal distribution in which the overall distribution contains a population of small clones and a population 20 times as large, respectively. FIGS. 4D-4G illustrate comparison of species richness estimates by Recon (middle) and CE (right) shown as in the lower panels of FIG. 3A for example additional gold standard overall distributions (left) for: a steep exponential clone-size distribution, a shallow exponential clone-size distribution, a bimodal distribution in which the overall distribution contains a population of small clones and a population 31 times as large, and a bimodal distribution in which the overall distribution contains a population of small clones and a population 20 times as large, respectively.

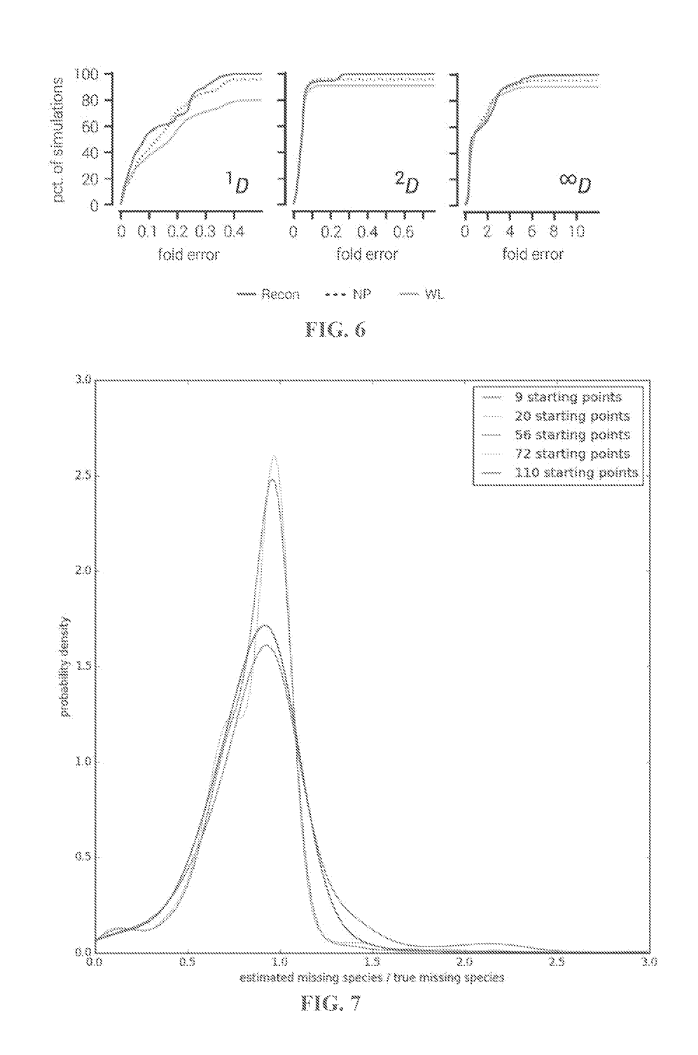

For species richness, Recon's estimates were found to be accurate to within 1% of the true diversity at 10× coverage, 10% at 3× coverage, and 50% at just 0.03× coverage—at which there may be just one cell in the sample for every 30 clones in the overall repertoire. Error for entropy and other weighted measures was lower. As shown in FIG. 3B, Recon (i.e., method 100) was robust to noise. Further illustrated in FIG. 3B and also in FIG. 6, Recon was found to be both faster and more accurate than the prior methods NP and WL, which like Recon may be used to estimate overall diversity by multiple diversity measures. For example, Recon's median runtime of 4.7 seconds (95^{th} percentile, 11 seconds) was >40× faster than WL and >800× faster than NP, both of which often took hours and sometimes days to complete. Recon's median error of 0.23× was smaller than that of NP (0.25×) and WL (0.26×), which was often off by orders of magnitude (mean, 198×; 95^{th} percentile, >1,500×). Recon was also more than twice as accurate as CE (0.53× median error).

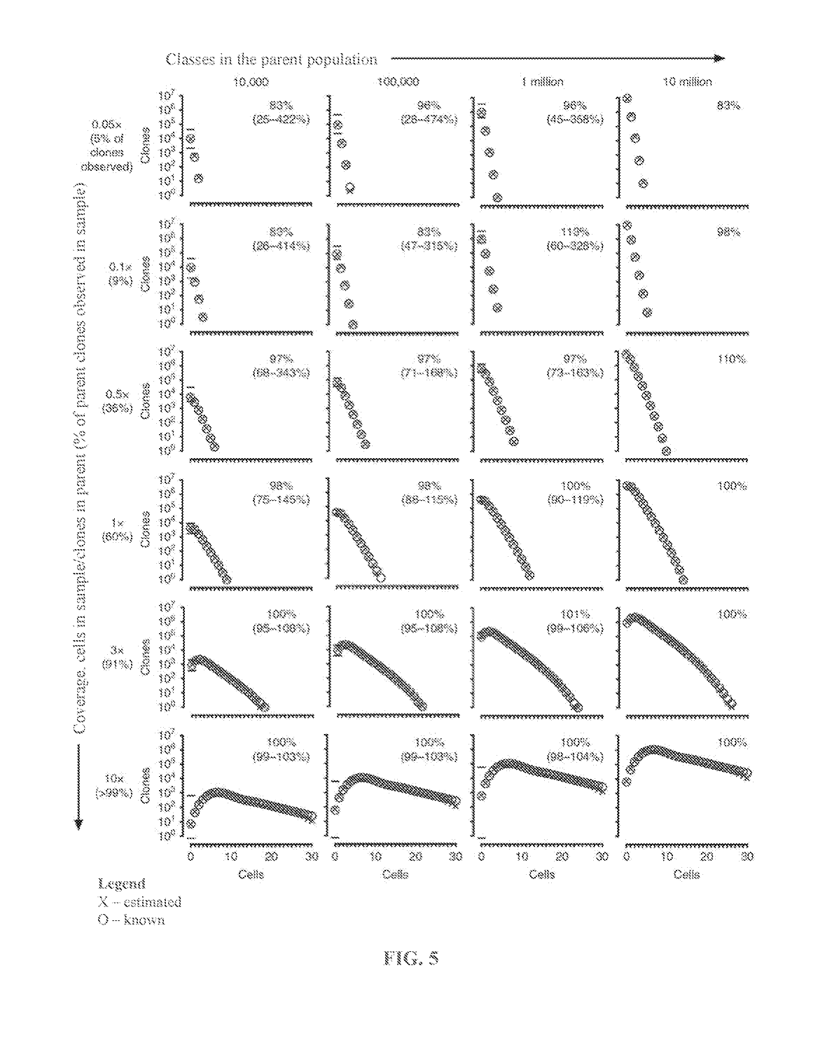

In this study self-consistency was also tested by resampling from the overall repertoires that were reconstructed from the gold-standard distributions to compare the resulting sample clone-size distributions to those of the original samples. As illustrated in FIG. 5, excellent agreement was found between predicted and observed frequencies of clone sizes across the range of overall diversities and levels coverage, including numbers of missing clones. FIG. 5 shows from left-to-right: overall distributions with increasing numbers of clones and from top-to-bottom: increasing sample size measured in coverage of the number of clones in the overall population. Open circles denote observed clone-size distributions, which was the input data given to Recon and crosses denote Recon's prediction of the clone-size distribution in the sample, based on its reconstruction of the clone-size distribution of the overall repertoire.

The benefit of using additional starting points in method 100 (Recon) was also studied based on probability densities of the ratio of estimated missing species/true missing species, as illustrated in FIG. 7. The study showed that a set of 56 starting points used may result in a sharper peak of the probability distribution function (pdf) centered near 1.0 (perfect accuracy), and diminished trapping in local minima away from 1.0. Pdfs were plotted using Gaussian kernel density estimates over 800 samples from gold-standard distributions.

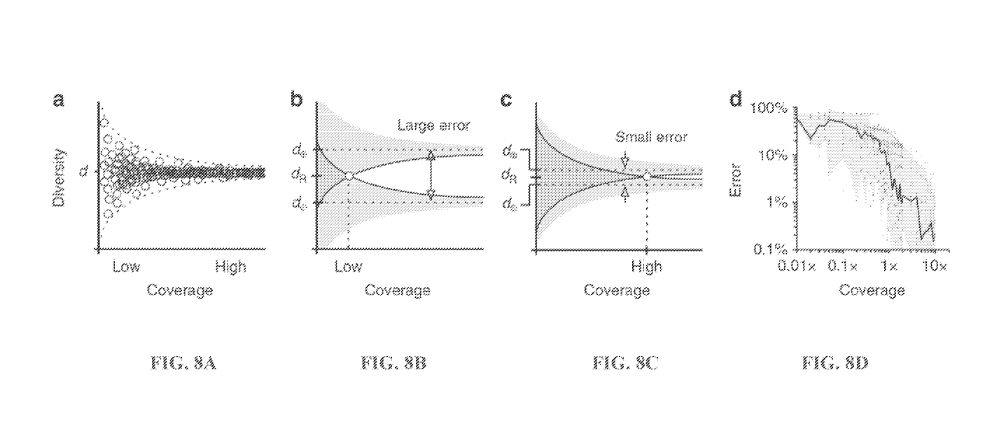

Error bars for the calculated diversity measures were also generated in this study. To generate error bars, each gold-standard repertoire was first sampled systematically at different levels of coverage (e.g., across three orders of magnitude of coverage 0.01×-10×). For each sample, the overall repertoire was reconstructed and the diversity of the reconstructed overall repertoire was determined as a function of coverage using method 100. Because higher coverage may produce more accurate estimates, the resulting error profile may converge with increasing coverage to the true overall diversity, as illustrated in FIG. 8A. The upper and lower bounds of this error profile may correspond to the largest and smallest values of estimated diversity, respectively, that are consistent with that repertoire's true diversity. In this way, an error profile was generated for the true diversity of each gold-standard repertoire. Separate profiles were generated for each diversity measure.

To find an error bar, using these error profiles, for a diversity measure of the reconstructed repertoire, the true diversity for which the diversity measure is at the lower bound, and the true diversity for which it is at the upper bound, were calculated as shown in FIGS. 8B or 8C. These true diversities define the upper and lower error bars, respectively. Combining error profiles across all samples suggested that ≥1× coverage may generally produce error bars of ≤10% for overall species richness as shown in FIG. 8D, which is consistent with result and analysis of FIGS. 3A and 3B.

Method 100 may use this error-bar framework to determine the coverage required to confidently detect differences in diversity between samples (e.g., between individuals or over time). Given an order-of-magnitude estimate of the overall diversity for two samples, method 100 may output the minimum sample size for which error bars for overall diversity estimates from these samples would not overlap, at detection thresholds ranging from e.g. 1.1× to 5× (Table 3 shown below). This sample size may be the minimum required to reject the null hypothesis that two estimates that differ by a given amount are actually from the same overall repertoire, at a confidence level of p=0.05 in an embodiment. For a given overall diversity there may be a minimum sample size below which the number of non-singlets is expected to be too small for method 100 to run. A study designed to detect a 1.1× (10%) difference in species richness between two samples, in which the samples are drawn from overall repertoires that have ˜100,000 clones, may require ≥313,792 cells from each sample for analysis. This is the number of cells in the sample that may be in small (e.g., ≤30 cells) clones that method 100 may require to perform reconstruction; if half of the cells in a sample of 314,000 cells belong to a single large clone, e.g. because of leukemia, the remaining half comprising the non-leukemic clones may be sufficient to detect a 20% difference in the species richness of the non-leukemic portion of the repertoire (which may require ≥153,543 cells), but not 10%.

Method 100 and the error-bar framework were also tested beyond exponentially and multimodally distributed samples. They were tested on a sample distribution previously identified as causing difficulties for overall species-richness estimation by multiple existing methods, corresponding to an overall population of ˜3,000 species sampled at ˜0.8× coverage. Three- and four-point mixture models, a logit normal model, a log-gamma model, and a beta model gave variable estimates that ranged from 2,930-3,494 overall species, with non-overlapping error bars that ranged from 2,867 to >10,000. In contrast, method 100 returned an estimate of 3,014 overall species, with error bars (2,709-3,513) that bracketed the range of other models' estimates, suggesting method 100 may provide improvement over multiple methods beyond WL and NP in arbitrary and/or difficult cases.

### Experiment #2

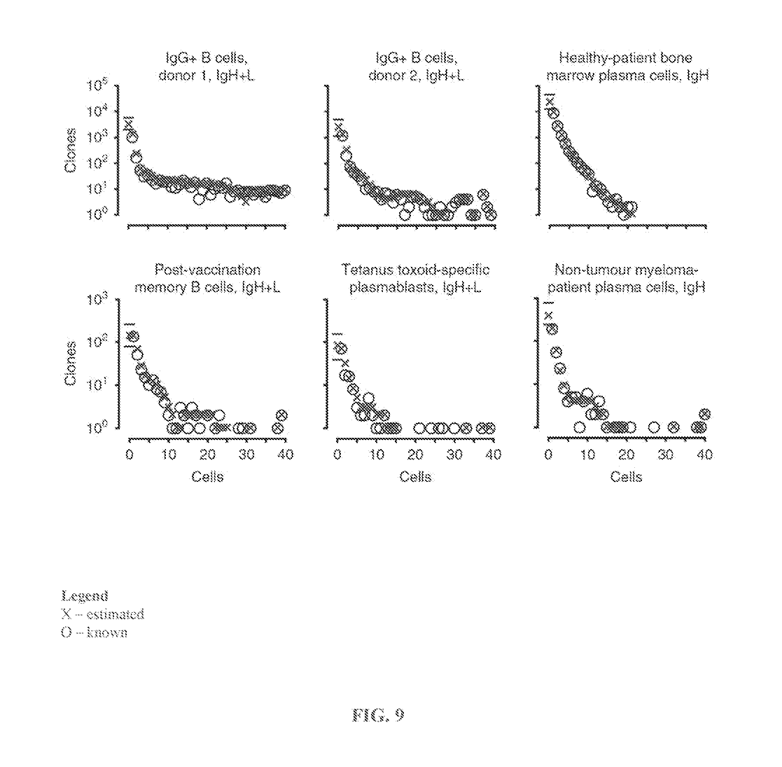

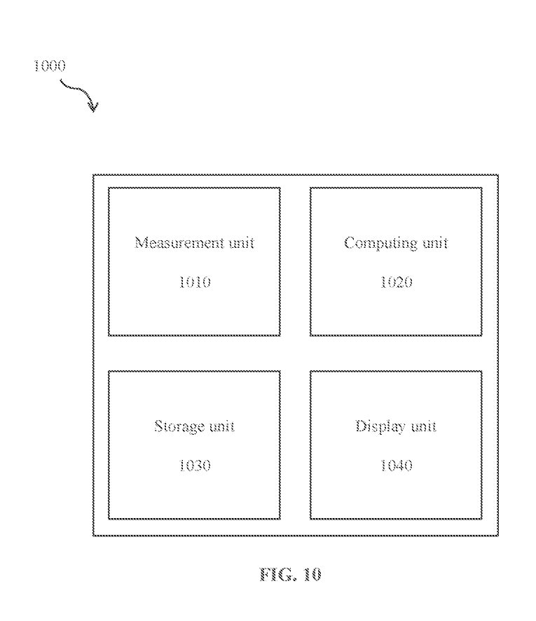

A study was performed with six different immune repertoires (listed below under a “subset column” of Table 4) for which sample distributions (e.g., clone-size distributions of samples) of each repertoire were known and publically available. Using a parent population reconstruction method, for example method 100, a maximum-likelihood estimate of the parent population of each immune repertoire was determined. The estimated parent populations were sampled to determine estimated sample distributions. These estimated sample distributions were then compared with the known sample distributions of the six immune repertoires, as illustrated in FIG. 9, which shows excellent agreement between the estimated and known clone-size distributions of samples for all six immune repertoires. The estimated and known clone sizes generally differed by less than 10 percent.

In this study, diversity measures, such as species richness and entropy were estimated using, for example, method 100 for the six immune repertoires, as shown in Table 4.

TABLE 4

| | | | | | | | | | Min | Strict |
|---|---|---|---|---|---|---|---|---|---|---|
| | | | | Species | | | Entropy | | clone | upper |
| | | | | richness | | Missing | (eff. no.) | | size, | bound, |
| Subset | Source | Method | Cells | Sample | Overall | species | Sample | Overall | cells | clones |
| IgG⁺ B cells, | healthy | IgH + L | 61,000 | 2,759 | 5,870 | 3,111 | 696 | 700 | 400 | 1 |
| individual 1 | adult | single | | | (4,761- | (2,002- | | (691- | | million |
| | | cell | | | 8,395) | 5,636) | | 720) | | |
| IgG⁺ B cells, | healthy | IgH + L | 47,000 | 2,211 | 4,616 | 2,405 | 345 | 348 | 700 | 5 |
| individual 2 | adult | single- | | | (3,374- | (1,163- | | (327- | | million |
| | | cell | | | 7,000) | 4,789) | | 373) | | |
| memory B | healthy | IgH + L | 8,000 | 336 | 473 | 137 | 21 | 21 | 30,000 | 14 |
| cells (IgG, | adult | single- | | | (446- | (77-245) | | | | million |
| IgM, and IgA) | vaccinee | cell | | | 614) | | | | | |
| tetanus | healthy | IgH + L | 2,000 | 159 | 239 | 80 | 3.5 | 3.5 | 1,000 | 300,000 |
| toxoid- | immunized | single- | | | (200- | (41-154) | | | | |
| specific | adult | cell | | | 313) | | | | | |
| plasmablasts | | | | | | | | | | |
| bone-marrow | healthy | IgH | 26,000 | 14,337 | 37,110 | 22,773 | 11,148 | 21,582 | 80 | 4 |
| plasma cells | adult | pooled | | | (27,350- | (13,013- | | (20,891- | | million |
| | | DNA | | | 58,916) | 44,579) | | 22,572) | | |
| non-tumor | multiple | IgH | 30,000 | 325 | 703 | 378 | 1.4 | 1.4 | 80 | 80,000 |
| plasma cells | myeloma | pooled | | | (563- | (238- | | | | |
| | patient | DNA | | | 1,081) | 756) | | | | |

### An Apparatus for Generating a Statistical Description of a System According to an Embodiment

FIG. 10 illustrates an apparatus 1000 for generating a statistical description of system, according to an embodiment. Apparatus 1000 may be used to implement one or more steps of method 100 as described above with reference to FIGS. 1 and 2, according to an example of this embodiment.

Apparatus 1000 may include a measurement unit 1010, a computing unit 1020, a storage unit 1030, and a display 1040, according to an embodiment. In an example, measurement unit 1010 may be configured to implement step 120 of method 100. For example, measurement unit 1010 may be configured to receive a sample of a parent population and measure statistics of the sample population. The sample population may be provided to measurement unit 1010 as a sample of an immune cell population from a blood sample. Measurement unit 1010 may be further configured to output the statistics as, for example, a frequency distribution of the sample population. In an example of this embodiment, measurement unit 1010 may be a nucleic-acid sequencing device.

Computing unit 1020 may include one or more hardware microprocessor units and may be configured to implement one or more steps (e.g., steps 130-150. steps 205-260) of method 100, according to an example of this embodiment. Computing unit 1020 may be configured to receive the output from measurement unit 1010, perform computations of for example, the steps of method 100 based on this output, and display one or more of the computed results to display 1040, or to an external device, according to an embodiment.

Computing unit 1020 may also comprise a field-programmable gate array (FPGA) that includes configurable logic, according to an example of this embodiment. The configurable logic may be programmed to perform the various functions discussed above of method 100 using configuration code stored in storage unit 1030. Likewise, computing unit 1020 may be programmed via instructions stored in memory 1030. These instructions may include one or more of the steps described above with reference to FIGS. 1 and 2.

Storage unit 1030 may include any type of memory including, for example, random access memory (RAM), read-only memory (ROM), electrically-erasable programmable read-only memory (EEPROM), FLASH memory, magnetic memory, optical memory, etc. Furthermore, storage unit 1030 may include volatile and non-volatile memory. For example, storage unit 1030 may contain a set of coded instructions in non-volatile memory for programming computing unit 1020. One or more of the computed results of the one or more steps of method 100 may also be stored in either the volatile or non-volatile memory depending on how long retention is desired. In another example, storage unit 1030 may be used to save data related to the statistics of the sample population and/or parameters related to the distribution model of the parent population, as discussed above with reference to FIGS. 1 and 2.

In a further embodiment, display 1040 may be configured to provide a visual representation of data received from measurement unit 1010, computing unit 1020, and/or storage unit 1030. For example, display 1040 may be used to display the statistics measured of the sample population by measurement unit 1010. These statistics may be displayed as a class size frequency distribution of the sample population. In another example, display 1040 may be used to display the diversity measures and/or statistical distributions of the estimated parent population as discussed above. Display 1040 may utilize any of a number of different display technologies such as, for example, liquid crystal display (LCD), light emitting diode (LED), plasma or cathode ray tube (CRT).

It should be noted that apparatus 1000 is shown in FIG. 10 as including only four units 1010-1040 for the sake of simplicity. However, as would be understood by a person of skill in the art based on the description herein, apparatus 1000 may include any number of functional units and devices that are not shown here.

### Example Environments for Implementing Method 100

For purposes of illustrating the structure and operation of the invention, the invention has been described in the environment of the cells of the adaptive immune system of an individual. The invention, however, has wide application. Other applications for the invention include the following examples.

According to an embodiment, method 100 may be implemented in an example environment of products in a manufacturing factory, In this environment, method 100 may be used to generate one or more descriptive statistics of the classes of failure modes of manufactures produced by the manufacturing factory. The generated one or more descriptive statistics may be one or more diversity measures of the parent population of all of the manufactures produced by the manufacturing factory. Such diversity measures of the parent population may help to increase the information obtained from product testing in order to decrease the amount of products that may be lost to testing, while still ensuring quality control in the manufacturing factory.

According to another embodiment, method 100 may be implemented in an example environment of search queries in an internet company. In this environment, method 100 may be used to generate one or more descriptive statistics of the classes of search query handled by the internet company. The generated one or more descriptive statistics may be one or more diversity measures of the parent population of search queries handled by the internet company. Such diversity measures of the parent query population may help the internet company to estimate the resources required for handling customer demands.

Still other example environments for application of the invention include:
1. In global telecommunications and related industries signal processing is ubiquitous. In signal processing, continuous signals are encoded as digital signals through sampling. The samples are interpolated to reconstruct the continuous signals. The invention may be useful in this process;
2. In polling and market research services and business analytics, extrapolation is used from the frequencies in a sample (e.g., number of viewers of a certain show; number of customers for a certain online product) to yield frequencies in the overall population. The invention may provide more accurate and dependable extrapolations;
3. The World Wide Web (overall population) has at least 4.2 billion web pages that have different numbers of (in- and out-) links (classes). The ability to search and index these pages is a core function of internet search giants, advertisers, and social networking companies, who sample a selection of pages for information about the whole. The invention can improve that process;
4. Machine learning involves taking data (overall population) and choosing a “training set” (sample) on which to learn. The invention could speed learning by helping choose better training sets;
5. The internet (overall population) consists of over six billion computers and other devices that have different numbers of network connections (classes). Sampled frequency distributions show that most of these devices have a very small number of connections, while a few have a very large number. A company analyzing the growth of the network might “ping” a sample of these devices to map the structure of the overall network in the sample. The invention could be used to improve the accuracy and value of those measurements;
6. Power grids are similar in network topology to the Internet and World Wide Web, and so the invention can be similarly useful in dense monitoring of nodes, switches, and relays. In addition, the invention could prove useful in predicting from past data the frequency and distribution of network outages; and
7. Research in biomedicine, public health, and physics all involve sampling and extrapolation in ways that the invention could improve. For example, in the chemotherapeutics industry, the invention could determine how well frequencies of drug-resistant mutations in a cancer sample reflect their frequencies in the individual, how likely it is that the absence of a drug-resistant mutation in a sample indicates lack of that mutation in the individual, or how well frequencies of drug-resistant mutations in a sample of the population reflect their frequencies

### An Example Computer System

Various aspects of the present invention may be implemented in computer readable code (e.g., software or firmware), hardware, or a combination thereof. FIG. 11 is an illustration of an example computer system 1100 in which embodiments of the present invention, or portions thereof, can be implemented as computer-readable code. For example, method 100 can be implemented in computer system 1100. Various embodiments of the present invention are described in terms of this example computer system 1100, After reading this description, it will become apparent to a person skilled in the relevant art how to implement embodiments of the present invention using other computer systems and/or computer architectures.

Computer readable code can include, for example, general programming languages (such as C or C++), hardware description languages (HDL) such as, for example, Verilog HDL, VHDL, Altera HDL (AHDL), or other available programming and/or schematic capture tools (such as circuit capture tools). This computer readable code can be disposed in any known computer-usable medium including a semiconductor, magnetic disk, optical disk (such as CD-ROM, DVD-ROM). As such, the code can be transmitted over communication networks including the Internet. It is understood that the functions accomplished and/or structure provided by the systems and techniques described above can be represented in a memory.

Computer system 1100 includes one or more processors, such as processor 1104. Processor 1104 is connected to a communication infrastructure 1106 (e.g., a bus or network).

Computer system 1100 also includes a main memory 1108, such as random access memory (RAM), and may also include a secondary memory 1110. Secondary memory 1110 can include, for example, a hard disk drive 1112, a removable storage drive 1114, and/or a memory stick. Removable storage drive 1114 can include a floppy disk drive, a magnetic tape drive, an optical disk drive, a flash memory, or the like. The removable storage drive 1114 reads from and/or writes to a removable storage unit 1118 in a well-known manner. Removable storage unit 1118 can include a floppy disk, magnetic tape, optical disk, flash drive, etc. which is read by and written to by removable storage drive 1114. As will be appreciated by persons skilled in the relevant art, removable storage unit 1118 includes a computer-readable storage medium having stored therein computer software and/or data.

Computer system 1100 (optionally) includes a display interface 1102 (which can include input and output devices 1103 such as keyboards, mice, etc.) that forwards graphics, text, and other data from communication infrastructure 1106 (or from a frame buffer or other display memory, not shown) for display on display unit 1130.

In alternative implementations, secondary memory 1110 can include other devices for allowing computer programs or other instructions to be loaded into computer system 1100. Such devices can include, for example, a removable storage unit 1122 and an interface 1120. Examples of such devices include a program cartridge and cartridge interface (such as those found in video game devices), a removable memory chip (e.g., EPROM or PROM) and associated socket, and other removable storage units 1122 and interfaces 1120 which allow software and data to be transferred from the removable storage unit 1122 to computer system 1100.

Computer system 1100 can also include a communications interface 1124. Communications interface 1124 allows software and data to be transferred between computer system 1100 and external devices. Communications interface 1124 can include a modem, a network interface (such as an Ethernet card), a communications port, a PCMCIA slot and card, a wireless interface (e.g., Bluetooth or WiFi), or the like. Software and data transferred via communications interface 1124 are in the form of signals which may be electronic, electromagnetic, optical, or other signals capable of being received by communications interface 1124. These signals are provided to communications interface 1124 via a communications path 1126. Communications path 1126 carries signals and can be implemented using wire or cable, fiber optics, a phone line, a cellular phone link, a RF link or other communications channels.

The terms “computer program storage medium” and “computer-readable storage medium” are used herein to generally refer to non-transitory media such as removable storage unit 1118, removable storage unit 1122, and a hard disk installed in hard disk drive 1112. Computer program storage medium and computer-readable storage medium can also refer to memories, such as main memory 1108 and secondary memory 1110, which can be memory semiconductors (e.g., DRAMs, etc.). These computer program products provide software to computer system 1100.

Computer programs (also called computer control logic) are stored in main memory 1108 and/or secondary memory 1110. Computer programs may also be received via communications interface 1124. Such computer programs, when executed, enable computer system 1100 to implement embodiments of the present invention as discussed herein. In particular, the computer programs, when executed, enable processor 1104 to implement processes of embodiments of the present invention, such as the steps in method 100 discussed above. Where embodiments of the present invention are implemented using software, the software can be stored in a computer program product and loaded into computer system 1100 using, for example, removable storage drive 1114, interface 1120, hard drive 1112, or communications interface 1124.

Embodiments of the present invention are also directed to computer program products including software stored on any computer-readable storage medium. Such software, when executed in one or more data processing device(s), causes a data processing device(s) to operate as described herein. Embodiments of the present invention employ any computer-readable medium, known now or in the future. Examples of computer-readable storage mediums include, but are not limited to, non-transitory primary storage devices (e.g., any type of random access memory), and non-transitory secondary storage devices (e.g., hard drives, floppy disks, CD ROMS, ZIP disks, tapes, magnetic storage devices, optical storage devices, MEMS, nanotechnological storage devices, etc.).

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A method for generating a modified maximum-likelihood description of an individual's immune system having a parent population of immune cells of different clones of immune cells, the method comprising:
collecting a sample population of immune cells from the individual;
measuring a statistical distribution of the sample population of immune cells using a nucleic-acid sequencing device or mass spectrometer;
determining a modified maximum-likelihood estimate of the parent population based on the statistical distribution of the sample population to produce an estimated parent population, wherein the determining of the modified maximum-likelihood estimate comprises:
initializing fit of a statistical model of the parent population based on the sample population, and
iteratively optimizing the statistical model until best fit parameters of the statistical model are above a sampling noise threshold; and
calculating a diversity measure of the estimated parent population, wherein the diversity measure is a representation of the modified maximum-likelihood description of the individual's immune system.

2. The method of claim 1, further comprising generating an error bar for the calculated diversity measure of the estimated parent population.

3. The method of claim 2, wherein the generating of the error bar comprises comparing a diversity measure of the estimated parent population and a diversity measure of a known parent population.

4. The method of claim 2, wherein the generating of the error bar comprises:
collecting a sample population from a known parent population;
determining a modified maximum-likelihood estimate of the known parent population based on the statistical distribution of the sample population of the known parent population to produce an estimated known parent population;
calculating a diversity measure of the estimated known parent population; and
comparing the diversity measure of the estimated parent population and the diversity measure of the estimated known parent population.

5. The method of claim 1, wherein the optimizing comprises adding parameters to the model in a first iterative process until the best fit parameters of the statistical model are above a sampling noise threshold.

6. The method of claim 5, wherein the optimizing comprises determining a maximum likelihood estimate of a set of initial parameter values of the model in a second iterative process, the second iterative process being performed during each iteration of the first iterative process.

7. The method of claim 6, wherein the determining of the maximum likelihood estimate of the set of initial parameter values of the model comprises:
selecting a set of initial parameter values from a plurality of sets of initial parameter values; and
determining, in a third iterative process, a modified maximum likelihood estimate of a number of missing clones in a sample population of the estimated parent population compared to the clones of the estimated parent population, the third iterative process being performed during each iteration of the second iterative process.

8. The method of claim 7, wherein the optimizing comprises determining, in a third iterative process, a maximum likelihood estimate of a number of missing clones in a sample population of the estimated parent population compared to the clones of the estimated parent population, the third iterative process being performed during each iteration of the second iterative process.

9. The method of claim 8, wherein the determining of the maximum likelihood estimate of the number of missing clones comprises:
estimating a number of missing clones;
maximizing a likelihood function;
outputting a new estimated number of missing clones; and
repeating the estimating, maximizing, and the outputting until the estimated number of missing clones is equal to the new estimated number of missing clones.

10. The method of claim 1, wherein the optimizing comprises:
approximating an initial parent population having same clone sizes;
computing, in an iteration of a first iterative process, a parent population having a number of clone sizes, the number being an integer equal to or greater than two;
incrementing the number by one in a subsequent iteration of the first iterative process when a predefined quality criterion is not met;
computing, in the subsequent iteration, a refined parent population having the incremented number of clone sizes; and
repeating the incrementing and the computing in the subsequent iteration until the predefined quality criterion is met.

11. The method of claim 1, wherein the estimated parent population is represented by a mixed Poisson model.

12. The method of claim 1, wherein the optimizing comprises determining parameter values that maximize a likelihood function of the statistical model.

13. The method of claim 1, wherein the determining is based on an expectation maximization algorithm.

14. The method of claim 1, wherein a sample population of the estimated parent population is a modified maximum-likelihood estimate of the sample population.

15. The method of claim 1, further comprising calculating a clone size of the parent population based on a clone size of the sample population.

16. An apparatus configured to generate a modified maximum-likelihood description of an individual's immune system having a parent population of immune cells of different clones of immune cells, the apparatus comprising:
a measurement unit configured to:
receive a sample population of immune cells from the individual, and
compute a statistical distribution of the sample population of immune cells; and a processing unit configured to determine a modified maximum-likelihood estimate of the parent population based on the statistical distribution of the sample population to produce an estimated parent population,
wherein, to determine the modified maximum-likelihood estimate of the parent population, the processing unit is further configured to:
initialize fit of a statistical model of the parent population based on the sample population; and
optimize the statistical until best fit parameters of the statistical model are above a sampling noise threshold.

17. The system of claim 16, wherein the processing unit is further configured to calculate a diversity measure of the estimated parent population, wherein the diversity measure is a representation of the modified maximum-likelihood description of the individual's immune system.

18. A computer program product stored on a non-transitory computer-readable medium, including instructions that, when executed by a computing device, cause the computing device to perform one or more operation comprising:
measuring a statistical distribution of a sample population of immune cells from an individual's immune system having a parent population of immune cells of different clones of immune cells;
determining a modified maximum-likelihood estimate of the parent population based on the statistical distribution of the sample population to produce an estimated parent population; and
calculating a diversity measure of the estimated parent population, wherein the diversity measure is a representation of a modified maximum-likelihood description of the individual's immune system.
